(19) United States
(12) Patent Application Publication
(10) Pub. No.: US 2022/0072201 A1
(43) Pub. Date: Mar. 10, 2022

(54) NASAL SEPTUM CARTILAGE COMPOSITIONS AND METHODS

(71) Applicant: Cook Biotech Incorporated, West Lafayette, IN (US)

(72) Inventors: Loran Solorio, West Lafayette, IN (US); Christopher Fecteau, Lafayette, IN (US); Will Hoggatt, West Lafayette, IN (US); Cameron Reese, Wake Forest, NC (US)

(73) Assignee: Cook Biotech Incorporated, West Lafayette, IN (US)

(21) Appl. No.: 17/454,673

(22) Filed: Nov. 12, 2021

Related U.S. Application Data
(63) Continuation of application No. PCT/US2020/034255, filed on May 22, 2020.
(60) Provisional application No. 62/852,636, filed on May 24, 2019.

(51) Int. Cl. A61L 27/36; A61L 27/44; A61L 27/28

(52) U.S. Cl. CPC A61L 27/3612; A61L 27/3687; A61L 27/28; A61L 27/44; A61L 27/3691

(57) ABSTRACT

Cartilage derived tissue compositions, methods of making, and methods of using same. The cartilage derived tissue compositions may comprise porcine nasal septal tissue. The cartilage derived tissue compositions also being processed to retain a beneficial component profile, while reducing cellular and DNA content.

### CROSS REFERENCE TO RELATED APPLICATIONS

This application is a continuation of PCT/US2020/034225 filed May 22, 2020 which claims the benefit of U.S. Provisional Application No. 62/852,636 filed May 24, 2019, which are hereby incorporated by reference.

### BACKGROUND

The present invention relates generally to implant materials, methods of making implant materials, as well as methods of their use. Native cartilage possesses a unique set of biochemical and ultrastructural characteristics, lending to its structural and mechanical functionalities within the body. Cartilage serves different purposes according to anatomical location, including providing structure to the nose and ears, and acting as a shock absorber and nearly frictionless gliding surface for motion within articulating joints. Hyaline cartilage is composed of sparse chondrocytes dispersed within a dense extracellular matrix containing glycosaminoglycans, which entrap water within the matric to impart compressive stiffness, and type II collagen, which lends tensile strength to the tissue, as well as a variety of growth factors. Due to the low cellularity and avascular nature of native cartilage tissue, cartilage is incapable of self-repair, and any damage occurring as a result of injury or disease typically leads to further degeneration.

Osteochondral autograft transfer, which involves the movement of small plugs of bone and cartilage from non-weight bearing regions of articular cartilage to a symptomatic lesion, is regularly used to treat defects in articular cartilage. However, osteochondral autograft transfer is limited to indications requiring small defects where concerns of donor site morbidity persist. Treatment of larger defects via osteochondral allografting is limited by the availability of healthy source tissue, and carries the additional risk of disease transmission. The complex molecular characteristics of cartilage further make the tissue a difficult tissue to re-create under in vitro conditions. In some of its aspects, the present disclosure is addressed to these needs.

### SUMMARY

In certain aspects the present disclosure provides unique tissue compositions. In accordance with some forms of the disclosure, provided is a cartilage-derived tissue composition comprising a cartilaginous tissue harvested from porcine nasal septum tissue. In certain embodiments the cartilaginous tissue has a DNA content of less than 50 μg/mg, and retains at least 30 μg/mg native sulfated glycosaminoglycans from the porcine nasal septum tissue source. In some forms, the cartilaginous tissue material has a DNA content of less than 25 μg/mg. In some forms the cartilage-derived tissue composition may include induced chemical crosslinks.

In certain embodiments, the cartilaginous tissue is a particulate cartilaginous tissue. In accordance with certain inventive variants the composition further comprises a polymeric matrix material, which may be a naturally derived polymeric matrix material. In some forms the present disclosure provides for a particulate cartilaginous tissue material intermixed with a polymeric matrix material, for example in a dry weight ratio of between 5:1 to 1:5.

In certain embodiments, the cartilaginous tissue material is present on a sheet-form substrate. In certain embodiments the sheet-form substrate comprises a sheet-form polymeric matrix material, which may be a naturally derived polymeric matrix material. When present on a sheet-form substrate the cartilaginous tissue material may be in sheet form or particulate form, and may optionally be intermixed with a polymeric matrix material.

In another aspect, provided is a method for preparing a cartilage-derived tissue composition, the method comprising: treating a cartilaginous tissue material harvested from porcine nasal septum tissue with a solution comprising hydrochloric acid (HCl) forming an HCl-treated cartilaginous tissue material having a DNA content less than 50 μg/g, and wherein the HCl-treated cartilaginous tissue material retains at least 30 μg/mg native sulfated glycosaminoglycans from the porcine nasal septum tissue source. In some forms the method also comprises removing perichondrium from the cartilaginous tissue. In certain embodiments the method further comprises grinding the HCl-treated cartilaginous tissue material to form a particulate.

Beneficial variants of the embodiments disclosed above in this Summary include those containing unique structural and/or functional features as described for the embodiments in the Detailed Description below.

Additional embodiments, as well as features advantages of aspects of the invention, will be apparent to persons of ordinary skill in the relevant art from the descriptions herein.

### BRIEF DESCRIPTION OF THE FIGURES

FIG. 1a is a 10× micrograph showing a cross section of an eSIS/pNSC SIS patch as described in Example 6 below incubated for 2 weeks in MSC media.

FIG. 1b is a 20× micrograph showing a cross section of an eSIS/pNSC SIS patch as described in Example 6 below incubated for 2 weeks in MSC media.

FIG. 2a is a 10× micrograph showing a cross section of an eSIS/pNSC SIS patch as described in Example 6 below incubated for 2 weeks in chondrogenic media.

FIG. 2b is a 20× micrograph showing a cross section of an eSIS/pNSC SIS patch as described in Example 6 below incubated for 2 weeks in chondrogenic media.

FIG. 3 is a PCR gel showing GAPDH present in each of the samples as described in Example 7 below, indicating cell growth in each sample.

FIG. 4 is a PCR gel showing increased production of COMP in the chondrocyte media after 40 cycles, as described in Example 7 below.

FIG. 5 is a PCR gel showing increased production of COL2 in the chondrocyte media after 40 cycles, as described in Example 7 below.

FIG. 6 is an image of a fine pNSC patch with MSCs grown on chondrogenic media for 1 week and stained using Safranin O, as described in Example 8 below.

FIG. 7 is an image of a commercially available collagen membrane with MSCs grown on chondrogenic media for 1 week and stained using Safranin O, as described in Example 8 below.

FIG. 8 is a graph illustrating the results of a comparative analysis of marrow-derived cells on either a commercially available collagen membrane or a fine pNSC patch as described in Example 8 below.

FIG. 9 is a graph illustrating the results of a comparative analysis of glycosaminoglycan production of a commercially available collagen membrane and a fine pNSC patch as described in Example 8 below.

### DETAILED DESCRIPTION

For the purposes of promoting an understanding of the principles of the invention, reference will now be made to certain embodiments thereof and specific language will be used to describe the same. It will nevertheless be understood that no limitation of the scope of the invention is thereby intended, and alterations and modifications in the referenced embodiments, and further applications of the principles of the invention as illustrated herein are contemplated as would normally occur to one skilled in the art to which the invention relates. In the discussions below, a number of features of a cartilaginous tissue material and/or methods for preparing a cartilaginous tissue material, and/or methods of using a cartilaginous tissue material are disclosed. It will be understood that any one, some or all of such disclosed features can be combined with the general embodiments discussed in the Summary above or set forth in the Listing of Certain Embodiments below to arrive at additional disclosed embodiments herein. Features disclosed herein are to be understood to be combinable with each other unless it is clearly stated or it is clear from context that they are not combinable.

As disclosed above, in certain aspects the present disclosure relates to compositions derived from a cartilaginous tissue source, method of making such compositions, as well as methods of using such compositions.

In certain embodiments, the present disclosure provides for compositions and methods of making compositions comprising cartilaginous tissue. In some forms the cartilaginous tissue is harvested from a warm blooded vertebrate. Mammalian cartilaginous tissues are preferred. In certain aspects the disclosure provides for cartilaginous tissues obtained from human donor tissues. In other embodiments the cartilaginous tissue may be obtained from, for example porcine, equine, bovine, ovine, and/or caprine sources. Porcine source tissue is particularly preferred for use in the presently disclosed composition and methods. Porcine nasal septum cartilage is particularly preferred in certain embodiments. In certain embodiments, the porcine nasal septal tissue is processed to remove non-cartilaginous tissues, for example the perichondrium may be removed prior to further processing of the nasal septal tissue material.

As used herein the terms “nasal septal tissue” and “nasal septum tissue” refer to the tissue portion which separates the nasal cavity into the two nostrils, in particular the cartilaginous portion, including the quadrangular cartilage, vomeronasal cartilage, lateral nasal cartilage, greater alar cartilage, and/or accessory nasal cartilage. The cartilaginous materials of the present disclosure are preferably derived from hyaline cartilage. The cartilage-derived tissue compositions of the present disclosure comprise collagen, preferably type-II collagen. In some forms the cartilage-derived tissue compositions of the present disclosure comprise predominantly type-II collagen.

In certain embodiments, the present disclosure provides cartilage-derived tissue compositions comprising glycosaminoglycans (GAG), including sulfated glycosaminoglycans (sGAG). GAG molecules are long chains of negatively charged polysaccharides that, along with type II collagen, comprise a critical component of the cartilage extracellular matrix (ECM). Certain embodiments of the present disclosure provide methods of making a material retaining native GAG and/or sGAG from a source tissue. Certain embodiments of the present disclosure provide methods of using a material as disclosed herein. As used herein the term glycosaminoglycan may refer to one or more of the following: heparin, heparin sulfate, chondroitin sulfate, dermatan sulfate, keratin sulfate, and/or hyaluronic acid. GAGs help guide cellular differentiation, as their strong negative charge binds positively charged growth factors. GAGs are highly polar and attract water, thus increasing the lubricity and shock absorbance of the cartilage-derived tissue composition as disclosed herein. As discussed herein, a Dimethylmethylene Blue (DMMB) Assay may be utilized to quantify the sGAG content of the cartilage-derived tissue compositions of the present disclosure.

Transforming growth factor-β1 (TGF-β01) is a potent chondrogenic factor that stimulates production of cartilage-specific ECM molecules in both chondrocytes and mesenchymal stem cells (MSCs). TGF-β1 has an isoelectric point (IEP) of 9.5, indicating that it has a strong net positive charge under neutral or acidic pH conditions. Within the physiological environment, binding and retention of TGF-β1 from surrounding blood and marrow drives the local differentiation of invading cells on pNSC particulate or within constructs comprising pNSC. Thus in certain embodiments, pNSC compositions contain TGF-β1, and/or sequester patient TGF-β1 which encourages differentiation of patient cells to produce cartilage-specific ECM molecules.

Thus, in certain embodiments the present disclosure provides cartilage-derived compositions having chondrogenic effects. In certain embodiments the cartilage-derived tissue compositions of the present disclosure encourage chondrogenic differentiation of host cells. In certain embodiments, compositions of the present disclosure may be utilized to encourage chondrogenic differentiation of mesenchymal stem cells (MSCs), which may be exposed to the cartilage-derived tissue composition of the present disclosure ex vivo and/or upon implantation of the cartilage-derived composition.

The cartilage-derived tissue compositions of the present invention are derived from a source tissue including native sGAGs. In forms the source tissue has a native sGAG content on a dry weight basis of about 150 μg/mg to about 600 μg/mg, preferably about 175 μg/mg to about 500 μg/mg, even more preferably about 250 μg/mg to about 400 μg/mg. In accordance with the present disclosure the source tissue has a native sGAG content on a dry weight basis of at least about 150 μg/mg, preferably at least about 250 μg/mg, even more preferably at least about 350 μg/mg.

In certain embodiments, cartilage-derived tissue compositions of the present disclosure retain native sGAGs from a source tissue. In some forms the cartilage-derived tissue compositions retain about 10 μg/mg to about 200 μg/mg native sGAGs on a dry weight basis, preferably about 20 μg/mg to about 175 μg/mg, even more preferably about 30 μg/mg to about 150 μg/mg. In certain embodiments, cartilage-derived tissue compositions of the present disclosure retain about 30 μg/mg to about 80 μg/mg sGAGs native to the porcine nasal septal tissue source, on a dry weight basis. In an alternative embodiment, cartilage-derived tissue compositions of the present disclosure retain about 100 μg/mg to about 150 μg/mg sGAGs native to the porcine nasal septal tissue source, on a dry weight basis. In some forms, the cartilage-derived tissue compositions retain at least about 10 μg/mg sGAGs native to the porcine nasal septal tissue source, on a dry weight basis, preferably at least about 30 μg/mg sGAGs native to the porcine nasal septal tissue source, on a dry weight basis, even more preferably at least about 50 μg/mg, sGAGs native to the porcine nasal septal tissue source, on a dry weight basis. In accordance with certain inventive variants, the cartilage-derived tissue compositions of the present disclosure retain at least about 100 μg/mg sGAGs native to the porcine nasal septal tissue source, on a dry weight basis. Additionally or alternatively, the present disclosure provides cartilage-derived tissue compositions having low or depleted DNA content relative to that of a source tissue. In certain embodiments the source tissue has a DNA content, on a dry weight basis, of about 25 μg/g to about 500 μg/g, preferably about 50 μg/g to about 400 μg/g, even more preferably about 60 μg/g to about 350 μg/g. In certain embodiments the source tissue has a DNA content of at least about 25 μg/g, preferably about 50 μg/g, even more preferably at least about 100 μg/g. In certain embodiments, cartilage derived tissue compositions of the present disclosure have a DNA content, on a dry weight basis, of less than about 50 μg/g DNA native to the porcine nasal septal tissue source, preferably less than about 25 μg/g DNA native to the porcine nasal septal tissue source, more preferably less than about 10 μg/g DNA native to the porcine nasal septal tissue source, even more preferably less than about 2 μg/g DNA native to the porcine nasal septal tissue source. In some forms, the cartilage derived tissue composition of the present disclosure are devoid of measurable DNA native to the porcine nasal septal tissue source, as defined by the assays described herein. In accordance with some forms, the cartilage derived tissue composition of the present disclosure has a DNA content, on a dry weight basis, of about 0 μg/g to about 50 μg/g DNA native to the porcine nasal septal tissue source, preferably about 0.5 μg/g to about 25 μg/g DNA native to the porcine nasal septal tissue source, more preferably about 1 μg/g to about 10 μg/g DNA native to the porcine nasal septal tissue source, even more preferably about 1 μg/g to about 5 μg/g DNA native to the porcine nasal septal tissue source.

The cartilage-derived tissue composition of the present disclosure may be provided in any suitable form. In certain embodiments, the cartilage-derived tissue composition of the present disclosure is a particulate. A particulate cartilage-derived tissue composition may be provided in a mixture with one or more additional components as discussed herein. In certain embodiments, a source tissue for the cartilage-derived tissue composition is processed to form a particulate composition. This processing may be in the form of grinding, cutting, shearing, tearing, milling, or any other suitable technique for forming a particulate. In some forms, the particulate may have an average particle cross section in the range of about 300 to about 350 microns, in some forms the particulate may have an average particle cross section in the range of about 20 to about 200 microns.

In certain embodiments the cartilaginous tissue material is processed to form a particulate material. In certain preferred embodiments the cartilaginous tissue material is processed to form a particulate material prior to treatment with an acidic solution as discussed herein, although it is within the scope of the disclosure to provide a material which is processed to form a particulate, either further processed to form a relatively finer particulate, or from a nonparticulate form, which has already been treated with an acid solution as disclosed herein. In certain embodiments such processing includes grinding a lyophilized material. In some forms a grinder having a 250 μm collar is used. Ultimately particles having various sizes may be produced. For example in certain embodiments a particulate is formed comprising particles having an average maximum cross-sectional dimension of between about 200 μm and about 500 μm, preferably between about 250 μm to about 400 μm, more preferably between about 300 μm to about 350 μm. In still further embodiments, a hammer mill is utilized to produce a fine powder particulate having an average maximum cross-sectional dimension of between about 5 μm and about 400 μm, preferably between about 10 μm to about 300 μm, more preferably between about 20 μm to about 200 μm. In accordance with some forms the present disclosure provides a particulate cartilaginous tissue material comprising particles having an average maximum cross-sectional dimension of less than 300 μm, preferably less than 250 μm, even more preferably less than 200 μm. In accordance with certain embodiments, the cartilaginous tissue material is cryomilled, for example milled with liquid nitrogen.

In certain embodiments, the present disclosure provides compositions comprising a cartilaginous tissue material and a matrix material. In some forms, the matrix material may comprise a polymeric matrix material. In certain embodiments, the polymeric matrix material may be a naturally derived matrix material. Exemplary naturally derived polymeric matrix materials include: collagen, gelatin, fibrin, elastin, alginate and other extracellular matrix materials. In certain embodiments the naturally derived polymeric matrix may be hydrolyzed. Suitable polymeric matrix materials can be provided by collagenous extracellular matrix materials (ECMs) possessing biotropic properties, including in certain forms, angiogenic collagenous extracellular matrix materials. For example, suitable collagenous materials include ECMs such as submucosa, renal capsule membrane, amnion, dermal collagen, dura mater, pericardium, fascia lata, serosa, peritoneum or basement membrane layers, including liver basement membrane. These and other similar animal-derived tissue layers can be expanded and processed as described herein. Suitable submucosa materials for these purposes include, for instance, intestinal submucosa, including small intestinal submucosa, stomach submucosa, urinary bladder submucosa, and uterine submucosa. In certain embodiments, an extracellular matrix material may be an expanded extracellular matrix material as described in U.S. patent application Ser. No. 12/488,974 to Johnson et al., published as US Patent Application Publication No. US20090318934A1, Dec. 24, 2009, which is hereby incorporated by reference. The expanded extracellular matrix material, such as expanded small intestinal submucosal tissue (“eSIS”), can have been expanded by contact with an alkaline solution (e.g. at a pH of 8 or higher, or 9 or higher), for example a sodium hydroxide solution, under conditions that lead to a volumetric expansion of the extracellular matrix tissue material (e.g. a 20% or greater, or 100% or greater volumetric expansion).

In some forms, the matrix material may comprise an artificial, or synthetic, polymeric matrix material. Exemplary artificial polymeric matrix materials include: polyvinyl alcohol (PVA), polyurethane, polyethylene glycol (PEG), polyethylene oxide (PEO), polylactide (PLA), polylactic-co-glycolitic acid (PLGA), carboxymethyl cellulose (CMC), polydimethylsiloxane (PDMS), polycaprolactone (PCL) and/or copolymers thereof.

In certain aspects, the present disclosure provides methods of preparing a cartilage-derived tissue composition as described herein. In accordance with certain inventive variants, porcine nasal septal cartilage is harvested and the perichondrium is removed. In certain embodiments, the cartilage is frozen in high purity water (HPW). In certain embodiments, the frozen cartilage is formed into a particulate. For example, in some forms the frozen cartilage is formed into a particulate by grinding, cutting, milling, grating, or shredding the frozen cartilage. In some forms the cartilage remains frozen during the process, for example liquid nitrogen milling may be used. In some forms the particulate is refrozen after processing. A sample may be prepared comprising cartilage harvest from one, or more than one source tissue.

In accordance with some modes, the cartilaginous tissue material is exposed to a hypotonic buffer. In certain embodiments, the cartilaginous tissue material is immersed in the hypotonic buffer. In some forms, the hypotonic buffer comprises Tris base, for example 0.1M Tris base. In certain forms the hypotonic buffer has a basic pH, for example around pH 8.0. In certain embodiments, the cartilaginous tissue material is exposed to the hypotonic buffer for at least about 12 hours, and not more than 36 hours, preferably about 24 hours.

In certain embodiments, the disclosed methods include the step of treating a cartilaginous tissue material harvested from porcine nasal septal tissue with an acidic solution. In certain embodiments the acidic solution comprises a strong acid, preferably a protic acid. In certain preferred embodiments the disclosed methods comprise treating a cartilaginous tissue material as described herein with an acidic solution comprising hydrochloric acid (HCl) forming an HCl-treated cartilaginous tissue material. In certain embodiments, the present disclosure provides for treating a cartilaginous tissue material harvested from porcine nasal septal tissue with a solution comprising 0.05 M to 0.2M hydrochloric acid (HCl). Preferably the present disclosure provides for treating a cartilaginous tissue material harvested from porcine nasal septal tissue with a solution comprising 0.1 M hydrochloric acid (HCl). In some forms the cartilaginous tissue material may be exposed to the HCl solution for at least 2 hours, at least 6 hours, or at least 12 hours; additionally or alternatively, not more than 60 hours. In certain embodiments, the cartilaginous tissue material is exposed to the HCl solution for between about 12 and 36 hours, preferably about 24 hours.

In addition to the above, and in accordance with certain embodiments, the present disclosure provides for disinfection of the cartilaginous tissue materials, which may comprise virally inactivation and/or endotoxin reduction and removal. In certain embodiments, an HCl-treated cartilaginous tissue material is disinfected immediately after treatment with an HCl solution as described above. In alternative embodiments, an HCl treated cartilaginous tissue material may be stored and subsequently disinfected as described herein. In still further alternative embodiments, a cartilaginous tissue material may be disinfected prior to treatment with hydrochloric acid as described herein. In some forms, a cartilaginous tissue material is disinfected by exposure to peracetic acid (PAA). In some forms, the cartilaginous tissue material is washed in a PAA solution. In certain embodiments, the PAA solution may comprise between about 0.1% PAA and about 1% PAA, preferably about 0.3% PAA. In certain embodiments, the cartilaginous tissue material is exposed to the PAA solution for at least 30 minutes, but not more than 3 hours. In preferred embodiments the cartilaginous tissue material is exposed to the PAA solution for about 1 hour. After disinfection, the disinfected cartilaginous tissue material may be washed with high purity water. In certain embodiments multiple washes (e.g. at least 3, at least 4, or at least 5 washes) are used to remove remaining PAA solution.

In accordance with the above the present disclosure provides an advantageous cartilaginous tissue material which has not been treated with detergents. Thus in some forms the disclosure provides for a cartilaginous tissue material which is free from, or substantially free from detergent residue.

In certain embodiments, the cartilaginous tissue material is dried. Drying can be conducted sufficiently to stabilize the processed cartilaginous tissue material. The drying of the processed cartilaginous tissue material can involve lyophilization (or freeze drying) or vacuum drying at ambient or elevated temperatures.

In accordance with certain inventive variants, the present disclosure provides cartilage-derived tissue compositions comprising HCl-treated cartilaginous tissue material combined with one or more additional polymeric matrix materials as discussed above. In some forms, the HCl-treated cartilaginous tissue material and polymeric matrix material(s) are mixed in liquid form (e.g. slurry) and then dried. In certain embodiments a composite material is formed comprising a polymeric matrix material and an HCl-treated cartilaginous tissue material in a dry weight ratio of about 10:1 to about 1:10, preferably 5:1 to about 1:5, more preferably about 3:1 to about 1:1. In some forms, the polymeric matrix material and the HCl-treated cartilaginous tissue material are mixed in a dry weight ratio of about 2:1.

In certain embodiments a layered structure may be provided. For example, in certain embodiments a plurality (i.e. two or more) of layers of a cartilage-derived tissue material can be bonded or otherwise coupled together to form a multilaminate structure. In some forms one or more layers of a cartilage-derived tissue material may be bonded to one or more layers of a sheet-form polymeric matrix material as discussed herein. In certain illustrative embodiments a multilaminate structure is formed by forming a layer of HCl-treated cartilaginous tissue material combined with one or more additional polymeric matrix materials as discussed above, onto at least one side of a sheet form polymeric matrix material. In other embodiments a multilaminate structure is formed by forming a layer of HCl-treated cartilaginous tissue material onto at least one side of a sheet form polymeric matrix material. The layers of such constructs can be bonded together in any suitable fashion, including dehydrothermal bonding under heated, non-heated or lyophilization conditions, using adhesives, glues or other bonding agents, crosslinking with chemical agents as described herein, radiation (including UV radiation), or any combination of these with each other or other suitable methods.

In this way, the present disclosure provides for graft structures having distinct properties on opposing sides. For example, in certain embodiments the present disclosure provides for multilaminate devices comprising a first layer comprising cartilaginous tissue material as discussed herein and a second supportive layer. In certain embodiments the second supportive layer may comprise a polymeric matrix material as discussed herein. In some forms the first layer may exhibit increased porosity relative to the second layer. In certain embodiments the first layer may be enriched with certain growth factors and/or glycosaminoglycans relative to the second layer. In this way, the first layer encourages cellular invasion and ingrowth on a first side, while the second layer provides a supportive barrier on the second side. In certain embodiments, the first layer may have a different component profile than the second layer, for example the first layer may comprise increased levels of collagen, specifically collagen II as compared to the second layer.

In certain embodiments, the present disclosure also provides a sheet-form cartilaginous tissue material. In some forms the sheet form cartilaginous structure is obtained from a collagenous source tissue and is processed to retain the naturally occurring collagenous structure of the source material. For example, in certain embodiment the present disclosure provides for sheet-form cartilaginous tissue materials which have been cut, or sliced in sheet form from a source tissue. The source tissue can be any source tissue described herein, preferably nasal septal tissue, preferably porcine nasal septum tissue. Thus, in some forms the sheet-form cartilaginous tissue material retains its native collagenous structure.

In some embodiments, sheet-form cartilaginous tissue materials are prepared by cutting the sheet-form material from a cartilaginous source tissue. This can be done by any suitable means, for example using a mandolin slicer and/or a scalpel. In this way sheet-form strips of various sizes may be obtained. In certain embodiments the sheet-form material has a average thickness of between 0.5 to 10 mm, preferably 0.75 to 5 mm, even more preferably 1 to 2 mm. Sheet-form cartilaginous tissue structures as discussed herein may be further subjected to any of the processing steps outlined herein. For example a sheet-form cartilaginous tissue material may be further treated with an acidic solution and/or disinfected as discussed above.

Sheet-form cartilaginous tissue materials may be processed as described herein, without being reduced to particulate form. For example, a sheet-form material may be subjected to one or more of the following steps as discussed herein: viral inactivation, rinse(s), exposure to a solution comprising HCl, treatment with a hypotonic buffer, sterilization, and/or lyophilization.

In certain embodiments, cartilaginous tissue material, in any form, can be crosslinked. A cartilaginous tissue material can be crosslinked either before or after it is formed into a medical device, or both. Increasing the amount (or number) of crosslinkages within the material or between two or more layers of the material can be used to enhance its strength. However, when a remodelable material is used, the introduction of crosslinkages within the material may also affect its resorbability or remodelability. Consequently, in certain embodiments, a cartilaginous material will substantially retain its native level of crosslinking, or the amount of added crosslinkages within the medical device will be judiciously selected depending upon the desired treatment regime.

For use in the present invention, introduced crosslinking of the cartilaginous tissue material and/or cartilage-derived tissue may be achieved by photo-crosslinking techniques, or by the application of a crosslinking agent, such as by chemical crosslinkers, or by protein crosslinking induced by dehydration or other means. Chemical crosslinkers that may be used include for example aldehydes such as glutaraldehydes, diimides such as carbodiimides, e.g., 1-ethyl-3-(3-dimethylaminopropyl)carbodiimide hydrochloride (EDC), genipin, diisocyanates such as hexamethylene-diisocyanate, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

When a multi-layered laminate material is contemplated, the layers of the laminate can be crosslinked to bond multiple layers of a multi-layered medical material to one another. Cross-linking of multi-layered medical materials can also be catalyzed by exposing the matrix to UV radiation, by treating the collagen-based matrix with enzymes such as transglutaminase and lysyl oxidase, by photocrosslinking, and by exposure to any of the chemical crosslinkers disclosed here. Thus, crosslinking may be added to individual layers prior to coupling to one another, during coupling to one another, and/or after coupling to one another.

The medical materials, constructs and devices of the invention can be provided in sterile packaging suitable for medical materials and devices. Sterilization may be achieved, for example, by irradiation, ethylene oxide gas, or any other suitable sterilization technique, and the materials and other properties of the medical packaging will be selected accordingly.

In certain embodiments, the cartilage-derived tissue composition of the present disclosure may be used in a surgical procedure. As discussed herein the cartilage-derived tissue composition of the present disclosure may be used an as implant material designed for human implantation. It is within the scope of the disclosure however, that the cartilage-derived tissue composition of the present disclosure may be used as an implant material for non-human implantation, for example with canine, feline, or equine subjects.

In accordance with certain inventive variants, the present disclosure provides a method of performing micro fracture surgery. The disclosed method includes the step of implanting a cartilage-derived tissue composition as disclosure herein. In certain embodiments the cartilage-derived tissue composition comprises a particulate cartilage-derived tissue composition. In certain embodiments the particulate cartilage-derived tissue composition is present on one side of a sheet-form substrate. In some forms the method comprises implanting the particulate cartilage-derived tissue composition present on one side of a sheet-form substrate, such the particulate material contacts the tissue defect. In certain embodiments the method also comprises removing calcified cartilage from a target joint. In certain embodiment the method also comprises forming a plurality of fractures in the subchondral bone plate. These fractures may cause formation of a clot material at the target site formed by blood and/or bone marrow. In certain embodiments the cartilage-derived tissue composition of the present disclosure is applied to the subchondral bone, in contact with one or more fractures, and/or in contact with the clot material.

The cartilage-derived tissue compositions of the present disclosure may be applied in a variety of surgical procedures. Exemplary surgical procedures wherein one or more of the embodiments disclosed herein may be applied include but are not limited to: tympanic membrane graft, micro fracture surgery, structural repair of cartilaginous structures (e.g. cartilaginous structures of the ear, nose, or throat), laryngeal surgery, tracheal surgery, and/or meniscus surgery. In certain embodiments the cartilaginous-derived tissue compositions of the present disclosure may be applied as a bulking agent. Exemplary procedures wherein a bulking agent of the present disclosure may be applied include but are not limited to: urethral procedures, and vocal chord procedures.

The following specific Examples are provided to promote a further understanding of certain aspects of the present disclosure. It will be understood that these Examples are illustrative, and not limiting, in character.

### Example 1

### Preparation of Porcine Nasal Septum Cartilage

Porcine nasal septal cartilage (pNSC) was harvested and the perichondrium was stripped off. The stripped cartilage was stored in high purity water (HPW) and frozen. The frozen septal material was then grated into smaller pieces and refrozen. Septal material was collected from 12 animals and pooled. A portion of the pooled sample was left unprocessed to provide a control sample while another portion of the pooled sample was subjected to an HCl processing treatment.

To prepare the HCl-processed material, the coarsely ground cartilage was thawed before being immersed in a hypotonic buffer for twenty-four hours. After the buffer wash, the material was immersed in 0.1 M HCl for 24 hours. After HCl immersion the material was subjected to a 1 hour viral inactivation wash with 0.3% peracetic acid (PAA). The PAA was then removed from the material with multiple HPW rinses. Finally, the HCl-processed cartilage was frozen, lyophilized, and transferred with dry ice to a ZM200 mill with a 250 μm collar (Retsch) and ground into a powder. Further details on the HCl processing treatment can be found in Table 1.

TABLE 1
HCl processing of pNSC

| Step | Wash |
|---|---|
| 1 | 10 g of cartilage immersed in 100 mL of |
| | hypotonic buffer (0.1M Tris base, pH 8.0) |
| | at 37° C. with 100 rpm orbital shaking for |
| | 24 hours. |
| 2 | 10 g of cartilage immersed in 100 mL of |
| | 0.1M HCl at 37° C. with 100 rpm orbital |
| | shaking for 24 hours. |
| 3 | 10 g of cartilage immersed in 100 mL of |
| | HPW at 37° C. with 100 rpm orbital |
| | shaking for 15 minutes. |
| 4 | 10 g of cartilage immersed in 100 mL of |
| | 0.3% PAA solution at 37° C. with 100 rpm |
| | orbital shaking for 1 hour. |
| 5 | 10 g of cartilage immersed in 100 mL of |
| | HPW at 37° C. with 100 rpm orbital |
| | shaking for 15 minutes, rinse water |
| | discarded and replaced with fresh HPW |
| | three times for a total of four HPW rinses. |

For the unprocessed control sample pNSC material, the coarsely grated cartilage was frozen, lyophilized, and transferred with dry ice to a ZM200 mill with a 250 μm collar (Retsch) and ground into a powder.

### Example 2

### Sulfated Glycosaminoglycan Content of HCl-Processed Porcine Nasal Septum Cartilage

The sulfated glycosaminoglycan (sGAG) content of the HCl-processed and unprocessed pNSC samples as prepared in Example 1 was determined using the Dimethylmethylene Blue (DMMB) assay. Triplicate samples, each approximately 25 mg, were taken from both processed and unprocessed pNSC. The samples were placed in microfuge tubes and 180 μl of PBS was added. A 180 μl sample of HPW served as a negative control and blank solution. 20 μl of Proteinase K (600 units/ml HPW) was added to each sample before being briefly mixed with a vortex mixer. The sample tubes were then placed in a 56° C. incubator with constant orbital shaking and left to digest. After 120 minutes the samples were determined to be completely digested and cooled to room temperature. A 1:750 dilution (2 μL of digest in 1498 μL of HPW) of the unprocessed samples was used.

A heparin standard curve was created from a stock solution of 1200 μg/ml heparin sodium salt (0.0024 g in 2 mL HPW) diluted with HPW to concentrations of 12, 24, and 120 μg/ml. The DMMB solution was made by adding 95 ml of 0.1 M HCl to 500 ml HPW followed by the addition of 0.0162 g DMMB, 3.0446 g glycine, and 2.3739 g NaCl. The solution was then diluted to 1000 mL with HPW and stirred until completely dissolved. The pH was adjusted to 3.08 using 1 M NaOH and/or 1M HCl. The DMMB was prepared fresh before the assay and protected from exposure to light.

For each sample, control, and standard, 200 μL was aliquoted into three corresponding 15 mL centrifuge tubes. These served as sample triplicates. 2.5 mL of DMMB solution as prepared above was added to each tube and briefly mixed. 200 μL of the solution was immediately aliquoted into a 96-well microplate and the absorbance was read at 600 nm. The reported absorbance values were averaged for each set of triplicate aliquots. The average absorbance values were then blank corrected using the average absorbance of the HPW control. The blank corrected average absorbance values for the heparin standards were used to create a standard curve. The results of the assay, listed in Table 2, indicate retention of at least 19% of the original sGAG content after HCl processing.

TABLE 2
Average sGAG Content of Processed
and Unprocessed pNSC Samples.

| | Average sGAG |
|---|---|
| Sample Lot | (μg/mg) |
| Unprocessed Samples | 360 |
| HCl-Processed Samples | 70 |

### Example 3

### DNA Content of HCl-Processed Porcine Nasal Septum Cartilage

HCl-processed and unprocessed pNSC samples as prepared in Example 1 were obtained and analyzed to quantify the extent to which HCl-processing and ethylene oxide (EO) sterilization affected the DNA content of pNSC. A portion of the HCl-processed and unprocessed sample were EO sterilized. The DNA was extracted and purified from the pNSC samples using a DNeasy Blood and Tissue kit (Qiagen). The DNA content was then quantified using Quant-iT PicoGreen dsDNA Reagent (Thermo Fisher Scientific). The average DNA content of the pNSC samples is listed in Table 3.

TABLE 3
Average DNA Content of pNSC Samples

| | Avg. μg DNA/ |
|---|---|
| Sample | g pNSC |
| Nonsterile unprocessed pNSC | 332 |
| EO sterilized unprocessed pNSC | 65.6 |
| Nonsterile HCl-processed pNSC | 1.72 |
| EO sterilized HCl-processed pNSC | 1.22 |

### Example 4

### Preparation of eSIS pNSC Composition

1.5 g of expanded small intestine submucosa (eSIS) strips (1% w/v) were blended for one minute with 150 mL of 0.05M acetic acid to form a slurry. 0.125 g chondroitin sulfate was added to the slurry and blended for 20 seconds. 0.75 g of HCl-processed pNSC particulate, prepared as described in Example 1, was added to the mixture and blended for one minute. The resulting mixture was adjusted for pH (3.0-3.5) if necessary using HCl and stored in a sealed container at 4° C.

### Example 5

### Preparation of eSIS/pNSC SIS Patch

The eSIS/pNSC mixture prepared as described in Example 4 was applied to one side of a 2-layer lyophilized sheet of small intestine submucosa (SIS). The resulting sheet was then placed on a pre-cooled −40° C. shelf.

A portion of the patches prepared as described above were further processed to crosslink the materials. The patches were soaked in EDC solution (50 mM EDC in 90% aqueous EtOH) at room temperature for 24 hours. The patches were removed from the solution and soaked in HPW for 2 hours at room temperature then compressed to remove air bubbles and ensure that the rinse water had penetrated the patch. The patches were further soaked in HPW overnight (16-18 hrs) at 4° C. then transferred to fresh HPW and compressed/released multiple times. The rinsed patches were then lyophilized.

### Example 6

### Cell Attachment: eSIS/pNSC SIS Patch

eSIS/pNSC SIS patches prepared as described in Example 5 were pretreated with complete MSC basal media and then seeded with mesenchymal stem cells (MSCs) on the eSIS/pNSC coating side. After two days five of the patches were changed to chondrogenesis media. After two and three weeks of culture, the patches were fixed, embedded and sectioned. The sections were stained with Hematoxylin and Eosin (H&E) and examined. Cells were found to grow on the surface as well as inside of the eSIS/pNSC coating. It was determined that MSC will grow on and invade into the eSIS/pNSC coating. Further, the eSIS/pNSC coating did not inhibit MSC from growing in chondrogenesis media.

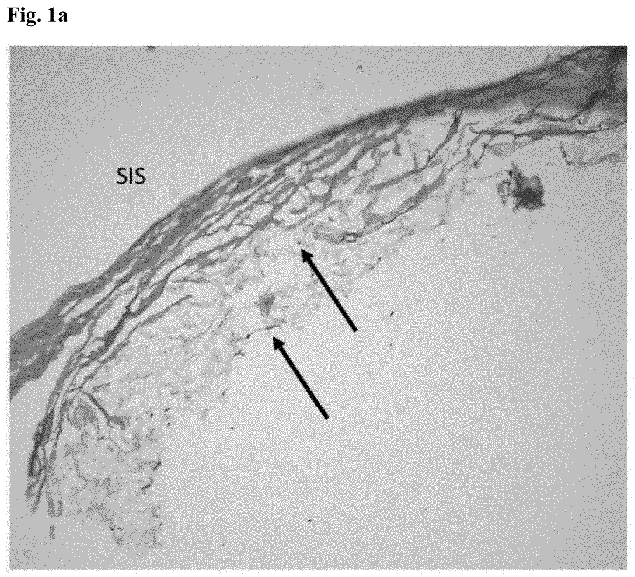

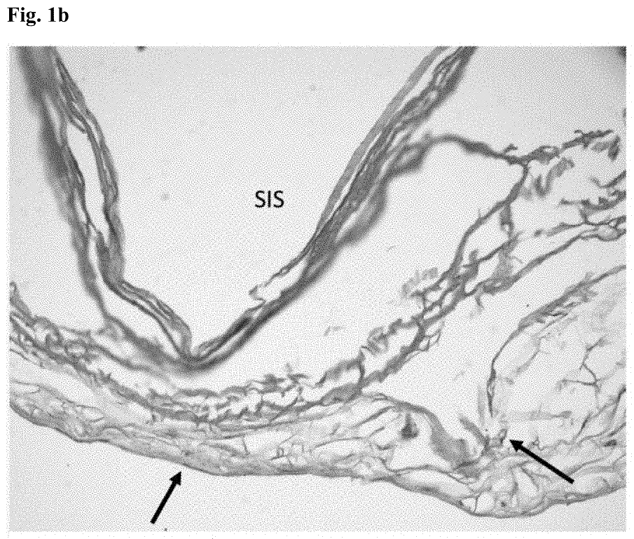

FIG. 1a is a 10× micrograph showing a cross section of an eSIS/pNSC SIS patch as described above incubated for 2 weeks in MSC media. FIG. 1b is a 20× micrograph showing a cross section of an eSIS/pNSC SIS patch as described above incubated for 2 weeks in MSC media. FIG. 2a is a 10× micrograph showing a cross section of an eSIS/pNSC SIS patch as described above incubated for 2 weeks in chondrogenic media. FIG. 2b is a 20× micrograph showing a cross section of an eSIS/pNSC SIS patch as described above incubated for 2 weeks in chondrogenic media. In each of FIGS. 1a, lb, 2a, and 2b the patch is comprised of an SIS portion and an eSIS/pNSC portion. Cells can be seen, for example as indicated by the arrows, within and upon the eSIS/pNSC portion of the patch.

### Example 7

### Chondrogenesis: eSIS/pNSC

Discs made from the eSIS/pNSC mixture as described in Example 4 were prepared by pipetting 800 μl of the eSIS/pNSC mixture into a mold and chilling at −40° C. A portion of the discs were crosslinked following the same procedure detailed in Example 5.

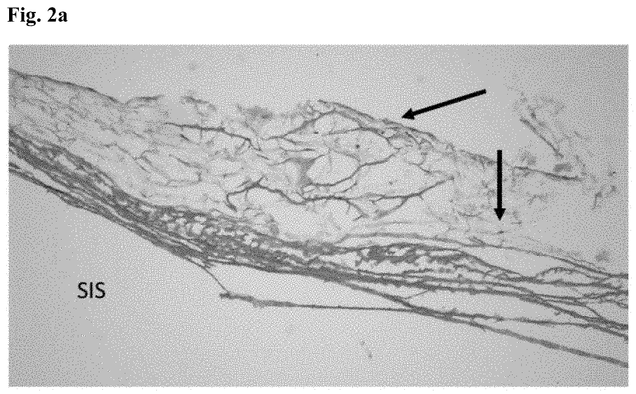

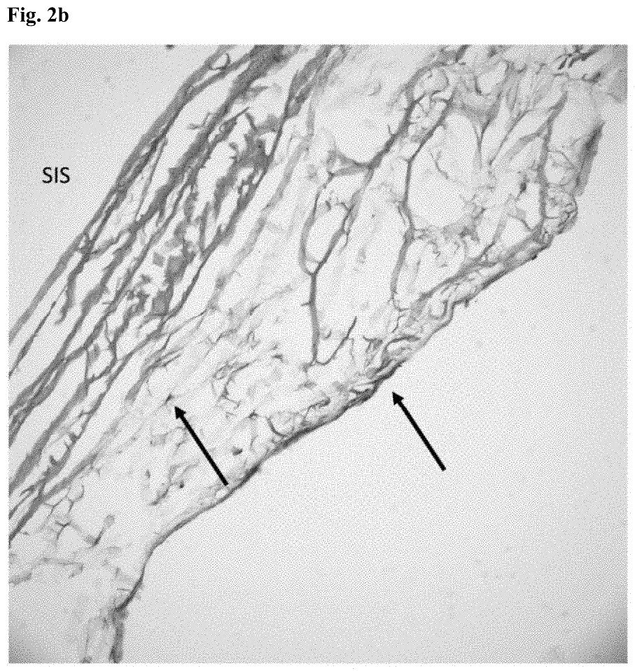

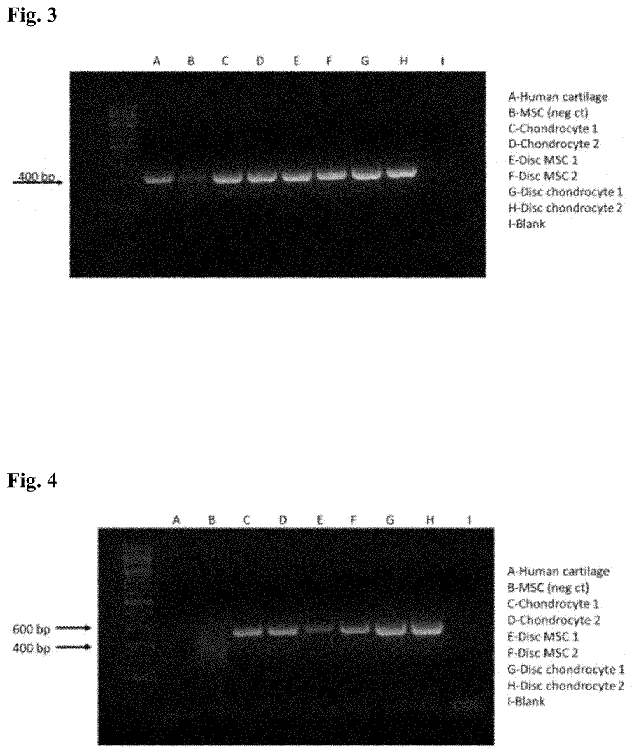

Human MSCs were seeded on both sides of eSIS/pNSC discs. After two days of culture, half of the discs were switched to chondrogenesis media. After a total of eighteen days of culture, total RNA was extracted. Qualitative RT-PCR was used to test for GAPDH as a housekeeping gene and Col2A1 and COMP as chondrogenic markers. Controls included chondrogenic pellet/spherical culture of MSC; MSC cultured on eSIS/pNSC discs in basal media; MSC cultured on tissue culture plastic in basal MSC media; and human cartilage. RT-PCR of total RNA demonstrated the presence of collagen II mRNA (COL2) in the MSC on the discs cultured in the MSC basal media. This study demonstrated that MSC will grow on the eSIS/pNSC discs and that the discs allow for MSC differentiation into chondrocytes. FIG. 3 is a PCR gel showing GAPDH present in each of the samples, indicating cell growth in each sample. FIG. 4 is a PCR gel showing increased production of COMP in the chondrocyte media after cycles. FIG. 5 is a PCR gel showing increased production of COL2 in the chondrocyte media after cycles.

### Example 8

### Chondrogenesis: eSIS/pNSC SIS Patch

eSIS/pNSC SIS patches were prepared as described in Example 5, except various lots were prepared using either “regular” or “fine” pNSC particulate. Regular pNSC is processed as described in Example 1. To obtain fine pNSC particulate (average maximum cross-sectional dimension of 20-200 μm) an HCl-processed cartilage material as described in Example 1 is obtained, the material is then frozen and cryomilled in a hammer mill with liquid nitrogen to obtain a fine particulate. 2-layer SIS was coated on one side with either the regular or fine eSIS/pNSC mixture and 10 mm punches were made and sterilized.

Human MSCs were cultured on the eSIS/pNSC SIS patches as well as on a commercially available collagen membrane patch. About 5×10⁵ cells were added to the eSIS/pNSC side of the patches and to the collagen membrane patch. All patches were cultured in complete basal media for three days. After three days a portion of the regular, fine, and collagen membrane patches were washed of the complete basal media and further cultured with complete chondrogenic media. The cells were cultured for 1 or 3 weeks, following which the patches were assayed for collagen II mRNA, sGAG, DNA, and examined histologically.

MSC on the eSIS/pNSC and collagen membrane discs were able to express collagen II mRNA after 3 weeks of culture, indicating that they were differentiating into chondrocytes. No collagen II mRNA was detected in the samples grown in basal media.

TABLE 4
Average μg sGAG/patch

| Sample | Media | 1-week | 3-week |
|---|---|---|---|
| Regular pNSC | Chondrogenic | 38.7 | 38.8 |
| Fine pNSC | Chondrogenic | 42.3 | 68.0 |
| Collagen membrane | Chondrogenic | 7.05 | 15.4 |
| Regular pNSC | Basal Media | 34.2 | 40.8 |
| Fine pNSC | Basal Media | 46.0 | 53.9 |
| Collagen membrane | Basal Media | 3.81 | 4.38 |
| Regular pNSC | No cells/media | 39.8 | |
| Fine pNSC | No cells/media | 47.4 | |
| Collagen membrane | No cells/media | 5.37 | |

TABLE 5
μg sGAG/patch secreted from cells

| Sample | Media | 1-week | 3-week |
|---|---|---|---|
| Regular pNSC | Chondrogenic | 0 | 0 |
| Fine pNSC | Chondrogenic | 0 | 20.6 |
| Collagen membrane | Chondrogenic | 1.68 | 10.0 |
| Regular pNSC | Basal Media | 0 | 1.0 |
| Fine pNSC | Basal Media | 0 | 6.5 |
| Collagen membrane | Basal Media | 0 | 0 |

TABLE 6
Average ng DNA/patch

| Sample | Media | 1-week | 3-week |
|---|---|---|---|
| Regular pNSC | Chondrogenic | 175 | 567 |
| Fine pNSC | Chondrogenic | 215 | 577 |
| Collagen membrane | Chondrogenic | 95.5 | 199 |
| Regular pNSC | Basal Media | 186 | 178 |
| Fine pNSC | Basal Media | 349 | 294 |
| Collagen membrane | Basal Media | 78.8 | 86.1 |
| Regular pNSC | No cells/media | 0.768 | |
| Fine pNSC | No cells/media | 9.43 | |
| Collagen membrane | No cells/media | 1.34 | |

TABLE 7
Average ng sGAG/ng DNA after removing basal levels of sGAG

| Sample | Media | 1-week | 3-week |
|---|---|---|---|
| Regular pNSC | Chondrogenic | 0 | 0 |
| Fine pNSC | Chondrogenic | 0 | 35.7 |
| Collagen membrane | Chondrogenic | 17.6 | 50.3 |
| Regular pNSC | Basal Media | 0 | 5.60 |
| Fine pNSC | Basal Media | 0 | 22.1 |
| Collagen membrane | Basal Media | 0 | 0 |

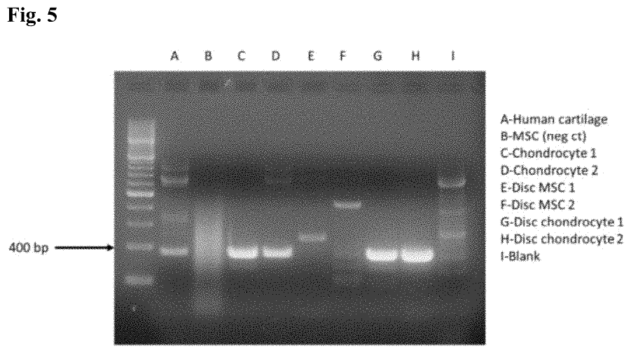

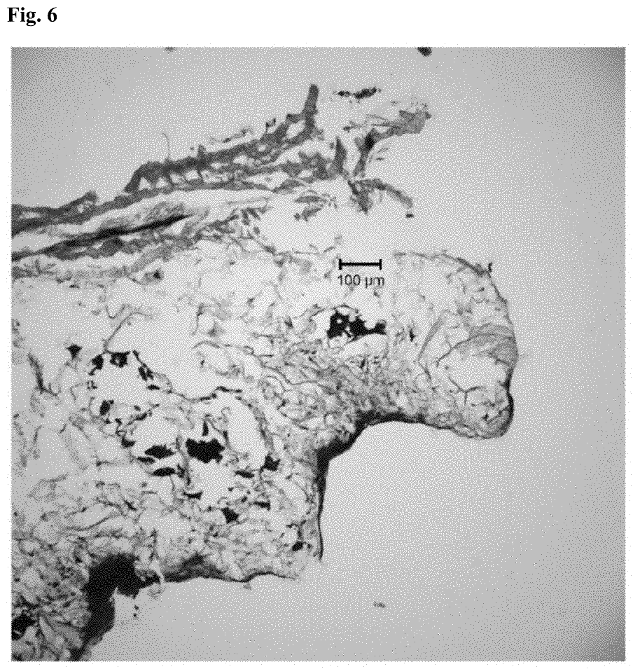

Histological analysis was done using H&E stain as detailed above, as well as Safranin O. FIG. 6 is an image of a fine pNSC patch with MSCs grown on chondrogenic media for 1 week and stained using Safranin O. FIG. 7 is an image of a commercially available collagen membrane with MSCs grown on chondrogenic media for 1 week and stained using Safranin O. The two samples (shown in FIGS. 6 and 7) have a different staining patterns indicating different component profiles, with red indicating cartilage and green indicating collagen.

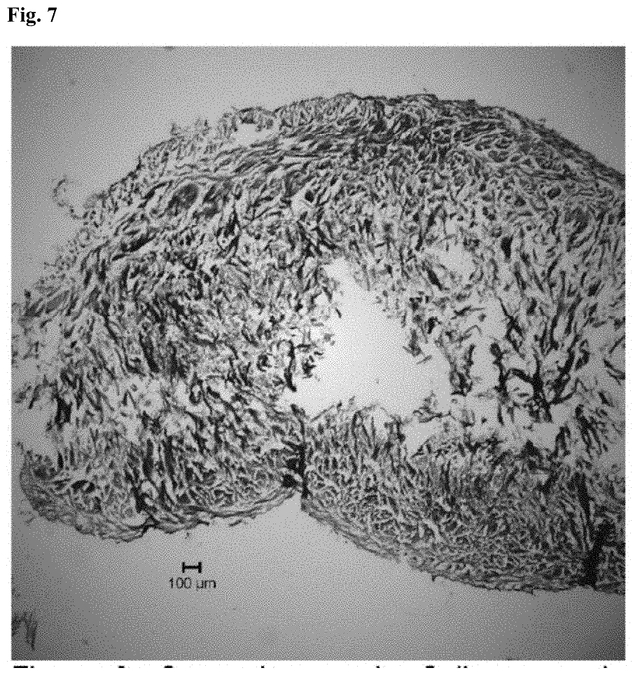

FIG. 8 is a graph illustrating the results of a comparative analysis of marrow-derived cells on either a commercially available collagen membrane or a fine pNSC patch as described herein. At both 1 week (blue) and 3 weeks (orange) the pNSC patch had increased cell retention and proliferation. Increasing the number of cells has been shown to benefit the quality of repair in microfracture surgery.

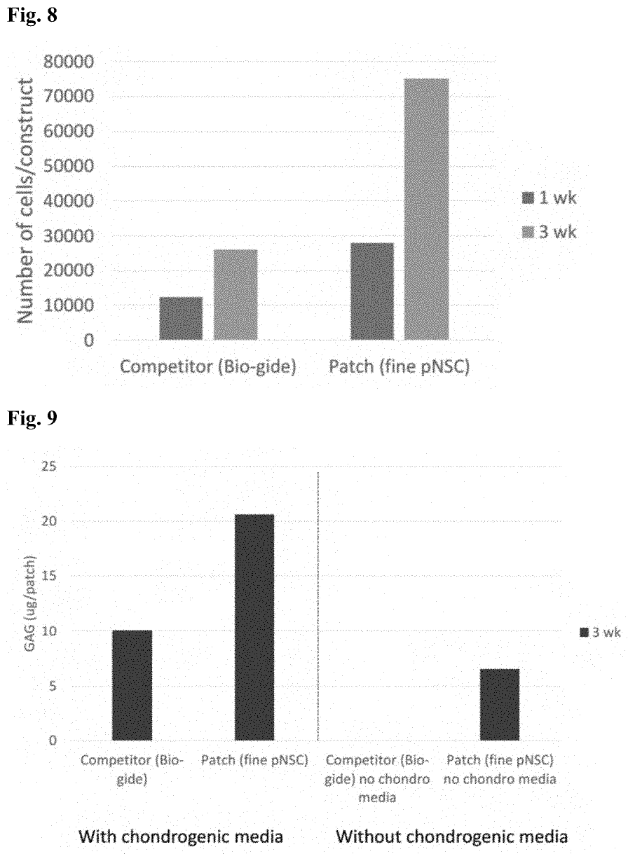

FIG. 9 is a graph illustrating the results of a comparative analysis of glycosaminoglycan production of a commercially available collagen membrane and a fine pNSC patch as described herein, both with and without addition of chondrogenic media (TGF-β1) after 3 weeks. The results show increased GAG production in the pNSC patch relative to the commercially available collagen membrane with chondrogenic media. The results also show that the pNSC patch is able to induce GAG productions, and thus chondrogenesis, without chondrogenic media, whereas the commercially available collagen membrane shows no GAG production without chondrogenic media.

### Example 9

### Production and Characterization of HCl-Processed pNSC Strips

Intact nasal septums were obtained from a porcine source and manually stripped of perichondrium. The septums were then sliced with a mandolin slicer or dissected with a scalpel to produce cartilage strips of various sizes with a average width of 1-2 mm. 10 g batches of tissue strips were processed as described in Example 1 without grinding, with a 1-hour viral inactivation treatment of 3% PAA. Following chemical processing and multiple HPW rinses, processed pNSC strips were produced. These strips were frozen at −80° C. for storage, and then thawed prior to DNA quantification by picoGreen assay and sGAG quantification by DMMB assay. Table 8 includes the DNA and sGAG results of HCl-processed and unprocessed pNSC strips.

TABLE 8
Average DNA and sGAG of wet pNSC Strips

| Sample | DNA ng/mg | sGAG μg/mg |
|---|---|---|
| HCl-Processed pNSC Strip (wet) | 0 | 60 |
| Unprocessed pNSC Strip (wet) | 86.7 | 201 |

### Example 10

### Effect of Peracetic Acid Concentration on pNSC

Ground pNSC material was processed according to Example 1, except that a portion of the material was treated with 0.3% PAA and a portion with 3.0% PAA. The materials were then analyzed for sGAG and DNA content in the same fashion as detailed in Example 2 and Example 3. A sample of 4-layer lyophilized SIS was also tested for GAG content. The results of the experiment are reported in Table 9.

TABLE 9
Effect of PAA Concentration on DNA and sGAG
levels in HCl-processed pNSC particulate.

| Sample | DNA ng/mg | sGAG μg/mg |
|---|---|---|
| 0.3% PAA | 21.5 ± 3.4 | 132.6 ± 5.0 |
| 3.0% PAA | 15.0 ± 4.2 | 94.6 ± 10.3 |
| SIS 2.0 | | 11.3 ± 1.4 |

### Example 11

### Net Surface Charge Characteristics of pNSC

Ground pNSC material was processed according to Example 1, except that a portion of the material was treated with 0.3% PAA and a portion with 3.0% PAA. The PAA treated pNSC material was frozen, cryogenically milled via liquid nitrogen-cooled hammermill, and lyophilized to dryness. Patch constructs incorporating either “low-PAA” (0.3% PAA) or “high-PAA” (3.0%) particulates were formed, as well as a sample comprising Small intestine sub mucosal (SIS) powder.

Zeta potential measurements of samples of low-PAA pNSC particulate, high-PAA pNSC particulate, and SIS particulate were analyzed on a Malvern Zetasizer Nano (N=3 measurements per sample). The results of the experiment are reported in table 10. The results confirmed that all samples tested had negative zeta potentials, with the magnitude of the negative charge directly correlated to the amount of GAG measured in each sample type.

TABLE 10
Effect of PAA Concentration on Net Charge (Zeta
Potential) in HCl-processed pNSC particulate.

| | Zeta Potential |
|---|---|
| Sample | (mV)) |
| 0.3% PAA | −25.6 ± 2.2 |
| 3.0% PAA | −20.3 ± 2.5 |
| SIS 2.0 | −10 ± 0.7 |

### Example 12

### Growth Factor Binding Characteristics of HCl-Processed pNSC Particulate

Samples of “low-PAA” pNSC particulate was determined to have the strongest negative charge, see Example 10 above. Thus samples of low-PAA pNSC particulate were analyzed both in particulate form as well as in patch constructs, for TGF-β1 adsorption. For each of the conditions shown below in table 11 20 mg samples (N=3) were incubated in 1 ml of a 20 ng/ml TGF-β1 solution. Following incubation for 16 hours at 37° C., the samples were briefly rinsed and the supernatants were assayed for unbound growth factor. As shown below in table 11, results demonstrated that the pNSC particulate and SIS powder both adsorbed and retained nearly all TGF-β1 from solution. The percent bound of each sample was calculated compared to the amount of TGF-β1 recovered from an aliquot of the initial 20 ng/ml solution (9.06 ng was recovered from the aliquot that was frozen, thawed, and assayed along with supernatants).

| | Avg. TGF-β1 | |
|---|---|---|
| Condition | in Supernatant | Percent Bound |
| Low PAA pNSC particulate | 0 | 100% |
| SIS | 0.008 | 99.9% |
| pNSC-incorporated patch | 0.740 | 91.8% |
| Collagen membrane | 0.366 | 96.0% |

### LISTING OF CERTAIN EMBODIMENTS

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.

Embodiment 1: A cartilage-derived tissue composition comprising:

a cartilaginous tissue material harvested from a porcine nasal septal tissue source, the cartilaginous tissue material having a less than 50 μg/mg DNA native to the porcine nasal septal tissue source on a dry weight basis, and wherein the cartilaginous tissue material retains at least 30 μg/mg sulfated glycosaminoglycans native to the porcine nasal septal tissue source on a dry weight basis.

Embodiment 2: The cartilage-derived tissue composition of embodiment 1, wherein said cartilaginous tissue material has a less than 25 μg/mg DNA native to the porcine nasal septal tissue source on a dry weight basis.
Embodiment 3: The cartilage-derived tissue composition of any one of the preceding embodiments, wherein said cartilaginous tissue material retains at least 50 μg/mg sulfated glycosaminoglycans native to the porcine nasal septal tissue source on a dry weight basis.
Embodiment 4: The cartilage-derived tissue composition of any one of the preceding embodiment, wherein said cartilaginous tissue is sterilized.
Embodiment 5: The cartilage-derived tissue composition of any one of the preceding embodiment, wherein said cartilaginous tissue is a particulate cartilaginous tissue.
Embodiment 6: The cartilage-derived tissue composition of embodiment 5, wherein said composition further comprises a polymeric matrix material.
Embodiment 7: The cartilage-derived tissue composition of embodiment 6, wherein said polymeric matrix material comprises a naturally derived polymeric matrix material.
Embodiment 8: The cartilage-derived tissue composition of any one of embodiments 6 or 7, wherein said particulate cartilaginous tissue material is intermixed with said polymeric matrix material.
Embodiment 9: The cartilage-derived tissue composition of any one of embodiments 6 to 8, wherein said particulate cartilaginous tissue material and said polymeric matrix material are present in a dry weight ratio in the range of 5:1 to 1:5.
Embodiment 10: The cartilage-derived tissue composition of embodiment 9, wherein said particulate cartilaginous tissue material and said polymeric matrix material are present in a dry weight ratio in the range of 3:1 to 1:1.
Embodiment 11: The cartilage-derived tissue composition of any one of the preceding embodiments, wherein the cartilaginous tissue material is present on a sheet-form substrate.
Embodiment 12. The cartilage-derived tissue composition of embodiment 11, wherein the sheet-form substrate comprises a sheet-form polymeric matrix material.
Embodiment 13: The cartilage-derived tissue composition of embodiment 12, wherein said polymeric matrix material is a naturally derived sheet-form polymeric matrix material.
Embodiment 14: The cartilage-derived tissue composition of any one of embodiment 1 to 4, wherein said cartilaginous tissue is in sheet-form.
Embodiment 15: The cartilage-derived tissue composition of embodiment 14, wherein the sheet-form cartilaginous tissue has an average thickness of 5 mm.
Embodiment 16: The cartilage-derived tissue composition of any one of the preceding embodiment, wherein said cartilage-derived tissue composition includes synthetically introduced chemical crosslinks.
Embodiment 17: A method for preparing a cartilage-derived tissue composition, the method comprising:

treating a cartilaginous tissue material harvested from a porcine nasal septal tissue source with an acidic solution to form an acid-treated cartilaginous tissue material having less than 50 μg/g DNA native to the porcine nasal septal tissue source on a dry weight basis, and wherein the acid-treated cartilaginous tissue material retains at least 30 μg/mg sulfated glycosaminoglycans native to the porcine nasal septal tissue source on a dry weight basis.

Embodiment 18: The method of embodiment 17, wherein the acidic solution comprises hydrochloric acid (HCl).
Embodiment 19: The method of embodiment 18, wherein the solution comprises hydrochloric acid at a concentration of 0.05 M to 0.2 M.
Embodiment 20: The method of any one of embodiment 17 to 19, further comprising:

removing perichondrium from said cartilaginous tissue material harvested from porcine nasal septal tissue.

Embodiment 21: The method of any one of embodiment 17 to 20, further comprising:

grinding the acid-treated cartilaginous tissue material to form a particulate prior to said treating with the acidic solution.

Embodiment 22: The method of any one of embodiment 17 to 21 further comprising:

treating the cartilaginous tissue material with a hypotonic buffer.

Embodiment 23: The method of any one of embodiment 17 to 22, further comprising:

treating the cartilaginous tissue material with viral inactivating solution.

Embodiment 24: The method of embodiment 23, wherein the viral inactivating solution comprises peracetic acid.
Embodiment 25: The method of any one of embodiment 17 to 24, further comprising:

sterilizing the cartilaginous tissue material.

Embodiment 26: The method of any one of embodiment 17 to 25, further comprising:

lyophilizing the cartilaginous tissue material.

Embodiment 27: The method of any one of embodiment 17 to 26, further comprising:

combining the acid-treated cartilaginous tissue material with a polymeric matrix material.

Embodiment 28: The method of embodiment 27, wherein said polymeric matrix material comprises a naturally derived polymeric matrix material.
Embodiment 29: A cartilage-derived tissue composition comprising:

a particulate material, the particulate material comprising particles of a cartilaginous tissue material harvested from a porcine nasal septal tissue source, and wherein said particles of cartilaginous tissue material have an average maximum cross-sectional dimension in the range of 10 μm to 300 μm.

Embodiment 30: The cartilage derived tissue composition of embodiment 29, wherein said particles of cartilaginous material have less than 50 μg/mg DNA native to the porcine nasal septal tissue source on a dry weight basis.
Embodiment 31: The cartilage derived tissue composition of any one of embodiments 29 or 30, wherein said particles of cartilaginous material retain at least 30 μg/mg sulfated glycosaminoglycans native to the porcine nasal septal tissue source on a dry weight basis.
Embodiment 32: The cartilage-derived tissue composition of any one of embodiment 29 to 31, wherein said particulate material further comprises a polymeric matrix material.
Embodiment 33: The cartilage-derived tissue composition of embodiment 32, wherein said polymeric matrix material comprises a naturally derived polymeric matrix material.
Embodiment 34: The cartilage-derived tissue composition of any one of embodiment 32 or 33, wherein said particulate cartilaginous tissue material is intermixed with said polymeric matrix material.
Embodiment 35: The cartilage-derived tissue composition of any one of embodiment 32 to 34, wherein said particulate cartilaginous tissue material and said polymeric matrix material are present in a dry weight ratio in the range of 5:1 to 1:5.
Embodiment 36: The cartilage-derived tissue composition of embodiment 35, wherein said particulate cartilaginous tissue material and said polymeric matrix material are present in a dry weight ratio in the range of 3:1 to 1:1.
Embodiment 37: The cartilage-derived tissue composition of any one of embodiment 29 to 36, wherein the cartilaginous tissue material is present on a sheet-form substrate.
Embodiment 38: The cartilage-derived tissue composition of embodiment 37, wherein the sheet-form substrate comprises a sheet-form polymeric matrix material.
Embodiment 39: The cartilage-derived tissue composition of embodiment 38, wherein said polymeric matrix material is a naturally derived sheet-form polymeric matrix material.
Embodiment 40: The cartilage-derived tissue composition of any one of embodiments 29 to 39, wherein said cartilage-derived tissue composition includes synthetically introduced chemical crosslinks.
Embodiment 41: A cartilage-derived tissue composition comprising:
a sheet-form cartilaginous tissue material harvested from a porcine nasal septal tissue source, and wherein the sheet-form cartilaginous tissue material has an average thickness of 5 mm, and wherein the sheet form cartilaginous tissue material retains at least 30 μg/mg sulfated glycosaminoglycans native to the porcine nasal septal tissue source on a dry weight basis.
Embodiment 42: The cartilage-derived tissue composition of embodiment 41, wherein said sheet-form cartilaginous tissue material has less than 50 μg/mg DNA native to the porcine nasal septal tissue source on a dry weight basis.
Embodiment 43: The cartilage-derived tissue composition of any one of embodiments 41 or 42, wherein the sheet-form cartilaginous tissue material is present in a multilaminate construct.
Embodiment 44: The cartilage-derived tissue composition of embodiment 43, wherein the multilaminate construct further comprises a sheet-form polymeric matrix material.
Embodiment 45: The cartilage-derived tissue composition of embodiment 44, wherein said polymeric matrix material is a naturally derived sheet-form polymeric matrix material.
Embodiment 46: The cartilage-derived tissue composition of any one of embodiment 41 to 45, wherein said cartilage-derived tissue composition includes synthetically introduced chemical crosslinks.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

1. A cartilage-derived tissue composition comprising:
a cartilaginous tissue material harvested from a porcine nasal septal tissue source, the cartilaginous tissue material having a less than 50 μg/mg DNA native to the porcine nasal septal tissue source on a dry weight basis, and wherein the cartilaginous tissue material retains at least 30 μg/mg sulfated glycosaminoglycans native to the porcine nasal septal tissue source on a dry weight basis.

2. The cartilage-derived tissue composition of claim 1, wherein said cartilaginous tissue material has a less than 25 μg/mg DNA native to the porcine nasal septal tissue source on a dry weight basis.

3. The cartilage-derived tissue composition of claim 1, wherein said cartilaginous tissue material retains at least 50 μg/mg sulfated glycosaminoglycans native to the porcine nasal septal tissue source on a dry weight basis.

4. (canceled)

5. The cartilage-derived tissue composition claim 1, wherein said cartilaginous tissue is a particulate cartilaginous tissue.

6. The cartilage-derived tissue composition of claim 5, wherein said composition further comprises a polymeric matrix material.

7. The cartilage-derived tissue composition of claim 6, wherein said polymeric matrix material comprises a naturally derived polymeric matrix material.

8. (canceled)

9. The cartilage-derived tissue composition of claim 6, wherein said particulate cartilaginous tissue material and said polymeric matrix material are present in a dry weight ratio in the range of 5:1 to 1:5.

10. (canceled)

11. The cartilage-derived tissue composition of claim 1, wherein the cartilaginous tissue material is present on a sheet-form polymeric matrix material substrate.

12. (canceled)

13. The cartilage-derived tissue composition of claim 11, wherein said sheet-form polymeric matrix material is a naturally derived sheet-form polymeric matrix material.

14. The cartilage-derived tissue composition of claim 1, wherein said cartilaginous tissue is in sheet-form.

15. The cartilage-derived tissue composition of claim 14, wherein the sheet-form cartilaginous tissue has an average thickness of 5 mm.

16. The cartilage-derived tissue composition of claim 1, wherein said cartilage-derived tissue composition includes synthetically introduced chemical crosslinks.

17. A method for preparing a cartilage-derived tissue composition, the method comprising:
treating a cartilaginous tissue material harvested from a porcine nasal septal tissue source with an acidic solution to form an acid-treated cartilaginous tissue material having less than 50 μg/g DNA native to the porcine nasal septal tissue source on a dry weight basis, and wherein the acid-treated cartilaginous tissue material retains at least 30 μg/mg sulfated glycosaminoglycans native to the porcine nasal septal tissue source on a dry weight basis.

18. (canceled)

19. The method of claim 17, wherein the acidic solution comprises hydrochloric acid at a concentration of 0.05 M to 0.2 M.

20. The method of claim 17, further comprising:
removing perichondrium from said cartilaginous tissue material harvested from porcine nasal septal tissue.

21. The method of claim 17, further comprising:
grinding the acid-treated cartilaginous tissue material to form a particulate prior to said treating with the acidic solution.

22. The method of claim 17, further comprising:
treating the cartilaginous tissue material with a hypotonic buffer.

23. The method of claim 17, further comprising:
treating the cartilaginous tissue material with viral inactivating solution.

24. The method of claim 23, wherein the viral inactivating solution comprises peracetic acid.

25. The method of claim 17, further comprising:
sterilizing the cartilaginous tissue material.

26. The method of claim 17, further comprising:
lyophilizing the cartilaginous tissue material.

27. The method of claim 17, further comprising:
combining the acid-treated cartilaginous tissue material with a polymeric matrix material.

28. The method of claim 27, wherein said polymeric matrix material comprises a naturally derived polymeric matrix material.

29. A cartilage-derived tissue composition comprising:
a particulate material, the particulate material comprising particles of a cartilaginous tissue material harvested from a porcine nasal septal tissue source, and wherein said particles of cartilaginous tissue material have an average maximum cross-sectional dimension in the range of 10 μm to 300 μm.

30. The cartilage derived tissue composition of claim 29, wherein said particles of cartilaginous material have less than 50 μg/mg DNA native to the porcine nasal septal tissue source on a dry weight basis; and
wherein said particles of cartilaginous material retain at least 30 μg/mg sulfated glycosaminoglycans native to the porcine nasal septal tissue source on a dry weight basis.

31. (canceled)

32. The cartilage-derived tissue composition of claim 29, wherein said particulate material further comprises a polymeric matrix material.

33. The cartilage-derived tissue composition of claim 32, wherein said polymeric matrix material comprises a naturally derived polymeric matrix material.

34. (canceled)

35. The cartilage-derived tissue composition of claim 32, wherein said particulate cartilaginous tissue material and said polymeric matrix material are present in a dry weight ratio in the range of 5:1 to 1:5.

36. (canceled)

37. The cartilage-derived tissue composition of claim 29, wherein the cartilaginous tissue material is present on a sheet-form polymeric matrix material substrate.

38. (canceled)

39. The cartilage-derived tissue composition of claim 37, wherein said polymeric matrix material is a naturally derived sheet-form polymeric matrix material.

40. The cartilage-derived tissue composition of claim 29, wherein said cartilage-derived tissue composition includes synthetically introduced chemical crosslinks.

41-42. (canceled)

43. The cartilage-derived tissue composition of claim 14, wherein the sheet-form cartilaginous tissue material is present in a multilaminate construct.

44. The cartilage-derived tissue composition of claim 43, wherein the multilaminate construct further comprises a sheet-form polymeric matrix material.

45. The cartilage-derived tissue composition of claim 44, wherein said polymeric matrix material is a naturally derived sheet-form polymeric matrix material.

46. (canceled)
